(19) United States
(12) Patent Application Publication
(10) Pub. No.: US 2005/0020678 A1
(43) Pub. Date: Jan. 27, 2005

(54) Environmentally safe fungicides

(76) Inventor: Robert Michael Denton, Delray Beach, FL (US)

Correspondence Address: STOCKWELL & ASSOCIATES, PSC, 861 CORPORATE DRIVE, SUITE 201, LEXINGTON, KY, 40503, US

(21) Appl. No.: 10/625,858

(22) Filed: Jul. 23, 2003

(51) Int. Cl. A61K 31/22; A01N 37/02

(52) U.S. Cl. 514/546

(57) ABSTRACT

An environmentally safe fungicide and its method of use are disclosed. The inventive environmentally safe fungicide comprises an effective concentration of an alkyl ester of lactic acid. Ethyl lactate is a preferred alkyl ester of lactic acid. Reduction in fungal contamination is achieved by applying the fungicide of the invention to a substrate to be decontaminated.

### CROSS-REFERENCE TO RELATED APPLICATIONS

This application claims priority from provisional application Ser. No. 60/397,655 filed Jul. 22, 2002.

### FIELD OF THE INVENTION

This invention relates to a method of using compositions of alkyl esters of lactic acid as non-toxic, environmentally safe fungicides. The fungicides are usable in a variety of contexts without harming plants, animals or humans.

### BACKGROUND OF THE INVENTION

Fungicides are widely used to eradicate fungi which may destroy ornamental and agricultural plants, crops, or trees; infect humans or animals; or cause the spoilage of foodstuffs. Although effective fungicides are known in the prior art, many are toxic or otherwise environmentally hazardous.

Several prior art fungicides may serve as examples Hexachlorobenzene may cause immune suppression in exposed animals. Phenols may cause severe symptoms in animals or humans such as nausea, vomiting, paralysis, coma or immune suppression. Formaldehyde is a skin irritant and carcinogen. Methyl bromide, the most widely applied agricultural fungicide in the world, destroys ozone molecules at 50 times the rate of the better-known chlorofluorocarbons.

The present invention relates to anti-fungal compositions comprising alkyl lactates, and the use of such compositions to treat foodstuffs, agricultural products, or animals, or alternatively, to treat surfaces which may contact foodstuffs, agricultural products, or animals.

It is a principal object of the present invention to provide an effective fungicide which eradicates various fungi from environmental surfaces, crops, trees, animals, and humans without producing adverse environmental effects.

### DETAILED DESCRIPTION OF THE INVENTION

It has been found by the inventor that compositions comprising alkyl lactates have surprising antifungal activity. The compositions of the invention comprise alkyl lactates wherein the alkyl group has 1 to i2 carbon atoms. Alkyl lactates are widely used in cosmetics and as food additives. Alkyl lactates are bio-degradable and non toxic. Accordingly, they are safe for topical use on plants or animals, or for the treatment of environmental surfaces to destroy fungi.

According to one preferred embodiment of the present invention, the fungicide contains ethyl lactate as the fungicidal ingredient. However, methyl, butyl, and propyl lactate may also be used. Fungicidal activity of the composition is attained at concentrations of the alkyl lactate greater than two per cent, achieving maximal fungicidal activity at alkyl lactate concentrations of approximately fifty percent. Accordingly, in one preferred embodiment, a highly effective composition is attained for safe, effective, direct topical application to humans and animals by employing between about two and fifty percent ethyl lactate.

In alternate embodiments, the fungicide may be formulated for application to plants, trees, crops, and other vegetation to provide a composition which effectively controls bothersome fungi, while being completely safe and non-injurious to the plant, tree, or crop, as well as to the flowers or fruit thereof. In addition, the compositions of this invention are completely biodegradable, and are safe to the environment and water supplies.

The composition of the invention may also contain one or several other compounds with antiseptic properties, perfumes, odorants, disinfectants, detergents or other additives and auxiliaries such as surfactants. The compositions according to the invention can be prepared by mixing the individual components together successively, if necessary with heating. No particular order need be adhered to during this process.

### EXAMPLES

The following example shows one preferred embodiment according to the invention. The components and specific ingredients are presented as being typical, and various modifications can be derived in view of the foregoing disclosure within the scope of the invention. A formulation is prepared as follows:

| Ingredient | Weight % |
|---|---|
| Ethyl Lactate | 16.0% |
| Fragrance | 1.0% |
| Water | 83.0%. |

Fragrance is added first to the ethyl lactate and then mixed in the water.

Turning to Tables 1 and 2 there is shown that the above formulation exhibits a high fungicidal efficiency.

TABLE 1
Results of challenge test using Penicillum pinopillum (ATCC 9644).

| | | |
|---|---|---|
| Challenge volume: | 5.0 | ml |
| Product volume: | 0.5 | ml |
| P. niger level of challenge liquid: | 0.4 | Million CFU/ml* |
| P. niger level of treated liquid: | 300 | CFU/ml |

| | |
|---|---|
| P. niger removal efficiency: | 99.25% |

*CFU/ml = Colony forming unit per milliliter.

TABLE 2
Results of challenge test using Aspergillus Niger (ATCC 9642).

| | | |
|---|---|---|
| Challenge volume: | 5.0 | ml |
| Product volume: | 0.5 | ml |
| A. Niger level of challenge liquid: | 0.9 | Million CFU/ml* |
| A. Niger level of treated liquid: | <1200 | CFU/ml |

| | |
|---|---|
| A. Niger removal efficiency: | 98.5% |

*CFU/ml = Colony forming unit per milliliter.

It is thus apparent from the results of the described Examples that the method of using compositions of alkyl lactates as fungicides according to the invention is a valuable method possessing surprisingly good fungicidal effects. The fungicides according to the present invention are well suited for a multitude of contexts, due to their environmentally friendly nature. The fungicides according to the invention are moreover easy to produce by a simple mixing process. Seen as a whole, therefore, the compositions according to the invention are especially suitable as fungicides.

One skilled in the art will appreciate that the particular alkyl lactate chosen can provide a more immediate and more effective reduction in fungal count depending on the fungi targeted. Accordingly, the described examples are merely exemplary and are in no way limiting.

One skilled in the art will also appreciate that the favored mode of applying the fungicides of the present invention to contaminated substrates may vary according to the-particular substrate. The fungicides of the present invention may be brought into direct physical contact with the substrates by wiping or spraying the fungicide, or alternatively, by dipping the substrate into the fungicide. These modes of application are mentioned by way of example and are no way limiting. The scope of this invention encompasses all modes of causing the fungicide to contact the contaminated substrate.

The concentration of alkyl lactate in accordance with the present invention should be sufficient to effect the desired reduction in fungal count over a reasonable time frame. One skilled in the art will recognize that concentration will depend upon a variety of factors, including the particular alkyl lactate employed, the targeted fungus, and the nature of the other compounds in the fungicide. Some fungi may require prolonged treatment involving multiple applications of compositions of the present invention. Suitable concentrations of alkyl lactate can be determined by conventional range-finding techniques known to those of ordinary skill in the art.

The invention has been described in detail with particular reference to preferred embodiments thereof, but it will be understood that variations and modifications can be effected within the spirit and scope of the invention.

1. An antifungal composition comprising at least one alkyl ester of lactic acid wherein the alkyl group of said alkyl ester has 1 to 12 carbon atoms.

2. The composition of claim 1 wherein the alkyl ester of lactic acid is methyl lactate.

3. The composition of claim 1 wherein the alkyl ester of lactic acid is butyl lactate.

4. The composition of claim 1 wherein the alkyl ester of lactic acid is propyl lactate.

5. The composition of claim 1 wherein the alkyl ester of lactic acid is ethyl lactate.

6. The composition of claim 2 wherein the concentration of methyl lactate is between about 2% and about 50% by volume.

7. The composition of claim 3 wherein the concentration of butyl lactate is between about 2% and about 50% by volume.

8. The composition of claim 4 wherein the concentration of propyl lactate is between about 2% and about 50% by volume.

9. The composition of claim 5 wherein the concentration of ethyl lactate is between about 2% and about 50% by volume.

10. The composition of claim 1 further comprising a composition selected from the group comprising water, perfumes, odorants, surfactants, disinfectants, detergents, or a combination thereof.

11. The composition of claim 2 further comprising a composition selected from the group comprising water, perfumes, odorants, surfactants, disinfectants, detergents, or a combination thereof.

12. The composition of claim 3 further comprising a composition selected from the group comprising water, perfumes, odorants, surfactants, disinfectants, detergents, or a combination thereof.

13. The composition of claim 4 further comprising a composition selected from the group comprising water, perfumes, odorants, surfactants, disinfectants, detergents, or a combination thereof.

14. The composition of claim 5 further comprising a composition selected from the group comprising water, perfumes, odorants, surfactants, disinfectants, detergents, or a combination thereof.

15. A method of reducing the fungal count on a substrate comprising applying the composition of claim 1 to the substrate.

16. The method of claim 15 wherein said substrate is selected from the group comprising a foodstuff, an agricultural product, a human or an animal, or a surface which may contact a foodstuff, an agricultural product, a human or an animal.

17. The method of claim 15 wherein said composition is applied to said substrate through a process selected from the group comprising dipping the substrate in said composition, spraying said composition on said substrate or wiping said composition on said substrate.

18. A method of reducing the fungal count on a substrate comprising applying the composition of claim 2 to the substrate.

19. A method of reducing the fungal count on a substrate comprising applying the composition of claim 3 to the substrate.

20. A method of reducing the fungal count on a substrate comprising applying the composition of claim 4 to the substrate.

21. A method of reducing the fungal count on a substrate comprising applying the antifungal composition of claim 5 to the substrate.

22. A method of reducing the fungal count on a substrate comprising applying the composition of claim 6 to the substrate.

23. A method of reducing the fungal count on a substrate comprising applying the composition of claim 7 to the substrate.

24. A method of reducing the fungal count on a substrate comprising applying the composition of claim 8 to the substrate.

25. A method of reducing the fungal count on a substrate comprising applying the composition of claim 9 to the substrate.

26. A method of reducing the fungal count on a substrate comprising applying the composition of claim 10 to the substrate.

27. A method of reducing the fungal count on a substrate comprising applying the composition of claim 11 to the substrate.

28. A method of reducing the fungal count on a substrate comprising applying the composition of claim 12 to the substrate.

29. A method of reducing the fungal count on a substrate comprising applying the composition of claim 13 to the substrate.

30. A method of reducing the fungal count on a substrate comprising applying the composition of claim 14 to the substrate.
